(19) United States
(12) Patent Application Publication
(10) Pub. No.: US 2005/0277602 A1
(43) Pub. Date: Dec. 15, 2005

(54) Methods employing agonists of P38 map kinase for the treatment of asthma

(76) Inventor: Michael M. Grunstein, Merion Station, PA (US)

Correspondence Address: Kathleen D. Rigaut, Ph.D.,J.D.;DANN, DORFMAN, HERRELL AND SKILLMAN, Suite 2400, 1601 Market Street, Philadelphia, PA, 19103, US

(21) Appl. No.: 11/135,221

(22) Filed: May 23, 2005

Related U.S. Application Data
(60) Provisional application No. 60/573,675, filed on May 21, 2004.

(51) Int. Cl. A61K 31/704; A61K 31/573

(52) U.S. Cl. 514/35; 514/171

(57) ABSTRACT

Methods for treating asthma using agonists of p38 are disclosed. Also provided are screening methods to identify test compounds which have efficacy for the treatment of asthma.

This application claims priority under 35 U.S.C. §119 (e) to U.S. Provisional Application 60/573,675 filed May 21, 2004, the entire contents of which are incorporated by reference herein.

Pursuant to 35 U.S.C. §202 (c), it is acknowledged that the U.S. Government has certain rights in the invention described herein, which was made in part with funds from the National Institutes of Health, Grant Numbers HL-31467 and HL-61038.

### FIELD OF THE INVENTION

The present invention relates the fields of medicine and signal transduction. More specifically, the invention provides methods for modulating MAP kinase-dependent signaling mechanisms implicated in the development of asthma.

### BACKGROUND OF THE INVENTION

Several publications and patent documents are cited throughout the specification in order to describe the state of the art to which this invention pertains. Each of these citations is incorporated herein by reference as though set forth in full.

Bronchial asthma in humans is characterized by inflammation of the airways, exaggerated airway reactivity to bronchoconstrictor agonists, and attenuated beta-adrenoceptor-mediated airway relaxation (Bai, 1990 Am. Rev. Respir. Dis. 141:552-557; Goldie et al., 1986, Br. J. Clin. Pharmacol. 22:669-676; McFadden et al., 1994, Am. J. Respir. Crit. Care Med. 150:523-526). In atopic asthma, mast cell activation has been implicated in mediating the immediate bronchoconstrictor response which acutely follows antigen inhalation. This response is a process which involves IgE-mediated activation of the high affinity IgE receptor (FcER1), leading to cellular degranulation and the release of various mast cell-derived mediators including histamine, eicosanoids, and specific cytokines (Metzger, 1992, Immunol. Rev. 125:37-48; Beaven et al., 1993, Immunol. Today 14:222-226; Galli, 1993, N. Engl. J. Med. 328:257-265). The identification of Fc receptors on other cell types in the lung (e.g., mononuclear cells, eosinophils, and dendritic cells) suggests that, apart from mast cells per se, these other cell types may also serve to propagate the pro-inflammatory allergic pulmonary response, most likely via the orchestrated extended release of various cytokines (Walker et al., 1992, Am. Rev. Respir. Dis. 146:109-115; Watson et al., 1993, Am. J. Respir. Cell Mol. Biol. 8:365-369; Capron et al., 1984, J. Immunol. 132:462-468; Beasley et al., 1989, Am. Rev. Respir. Dis. 139:806-817; Litchfield et al., 1992, J. Asthma 29:181-191; Barnes et al., 1988, Pharmacol. Rev. 40:49-84; Borish et al., 1991, J. Immunol. 146:63-67.

Mitogen-activated protein (MAP) kinase cascades are one of the most studied and elucidated signal transduction systems, and are known to participate in multiple directions of cellular programs (Puddicombe, et al. (2000) Clin. Exp. Allergy 30:7-11). Three key MAP kinase cascades have been described in mammalian cells, including the extracellular regulated kinase (ERK) 1 and 2, the c-Jun N-terminal kinase (JNK), and the p38 MAP kinase. These MAP kinases are dual phosphorylated on tyrosine/threonine residues by distinct MAP kinase kinases. ERK1/2 corresponding to classical MAP kinases are activated by a variety of growth factors and play critical roles in mitogenesis. JNK and p38 are activated by cellular stress or proinflammatory cytokines that are known to induce apoptosis. Several studies have shown the functional role of MAP kinases in bronchial epithelium using their specific inhibitors (Matsumoto et al. (1998) J. Allergy Clin. Immunol. 101: 825-831; Hashimoto et al. (2000) Clin. Exp. Allergy 30:48-55). According to these results, JNK and p38 MAP kinase play critical roles in regulating cytokine production by the cells activated by a range of stimuli. Additionally, MAP kinases have been shown to regulate cytokine gene expression in human airway smooth muscle cells (Hedges et al. (2000) Am. J. Respir. Cell Mol. Biol. 23:86-94.

Current treatment options for asthma include medications that control the airway inflammatory component of the disease, e.g., primarily corticosteroids, sodium cromolyn, methylxanthines, leukotriene modifiers) and rapid relief medications that counteract bronchospasm, e.g., primarily beta-adrenergic agents. There are several disadvantages to using these medications as follows. There is a potential lack of effective sustained action; there are side effects associated with prolonged use of these medications, particularly in the case of corticosteroids and beta-adrenergic agents; there is a progressive loss of sensitivity to these treatments after prolonged use; there is limited efficacy of any of these agents in severe cases of asthma; these agents are non-selective, i.e., they do not specifically target the lung, therefore, side-effects affecting other organs are a potential risk. Furthermore, there are data which document an increased risk of dying from bronchial asthma following prolonged treatment of asthma using long-acting beta-adrenergic agents such as fenoterol (Pearce et al., 1990, Thorax 45:170-175; Spitzer et al., 1992, N. Engl. J. Med. 326:560-561).

Approximately fifteen million individuals in the U.S. have asthma and the disease is the cause of more than five thousand deaths annually in the U.S. In children, asthma represents the most prevalent chronic disease, requiring the most frequent use of emergency room visits and hospitalizations. The overall annual cost for asthma care in the U.S. is estimated to be in the range of billions of dollars. Asthma is the most common cause of school and work absenteeism in the U.S.

There is thus a long felt need for additional and more specific and effective compositions and methods for treatment of asthma which additional compositions and methods overcome the deficiencies of the prior art compositions and methods.

### SUMMARY OF THE INVENTION

The present invention provides methods for the treatment of asthma. An exemplary method entails the administration of an effective amount of an agonist of p38 MAP kinase in a patient in need thereof to alleviate the symptoms of asthma. An exemplary agonist comprises anisomycin and derivatives thereof. Exemplary derivatives include 4-O-dodecanoyl-3-O-carbamoyldeacetylanisomycin; 3-O-methylcarbamoyl-deacetylanisomycin; 4-O-acetyl-3-O-carbamoyldeacetylanisomycin; 4-O-hexanoyl-3-O-carbamoyldeacetylanisomycin; 4-O-heptanoyl-3-O-carbamoyldeacetylanisomycin; 3-O-methoxymethyldeacetylanisomycin and 3-O-carbamoyldeacytylanisomycin. Preferred methods for the administration of such compounds include systemic administration and administration directly to airway smooth muscle cells.

In yet another aspect of the invention, the method entails administration of at least one additional agent known to efficacious for the treatment of asthma. Such agents include, without limitation, corticosteroids, sodium cromolyn, methylxanthines, leukotriene modifiers and rapid relief medications that counteract bronchospasm, e.g., beta-adrenergic agents.

Another aspect of the invention includes methods for screening and identifying test compounds which act as agonists of p38MAPK. An exemplary method entails providing cells which express p38 MAPK and exhibit an inflammatory response in response to challenge with a pro-asthmatic agent, challenging the cells with the agent in the presence and absence of the test compound; and determining the phosphorylation state of p38MAPK in the presence and absence of said test compound, compounds increasing the phosphorylation state of p38MAPK being identified as agonsts of p38MAPK. In a preferred embodiment, the cells are airway smooth muscle cells. The method can optionally entail measuring constrictor and relaxation responses of airway smooth muscle cells in the presence and absence of said test compound. In yet another aspect, the method encompasses determining the phosphorylation state of ERK1/2. Exemplary compounds useful in the method above include anisomycin and derivatives thereof. A co-culture system of antigen presenting cells and dendritic cells may also be employed to screen and identify agonists of p38 MAPK. Finally, the screening method may be performed in vitro in tissue culture or in vivo in whole animal models of allergic asthma.

### BRIEF DESCRIPTION OF THE DRAWINGS

FIG. 1 is a graph showing a comparison of constrictor responses to acetylcholine (Ach) in rabbit ASM tissues exposed for 24 h to Der p1 in the absence and presence of the cysteine protease inhibitor, E-64. Data represent mean±SE values from 8 experiments.

FIG. 2 is a graph showing a comparison of relaxation responses to isoproterenol in rabbit ASM tissues exposed for 24 h to Der p1 in the absence and presence of E-64. Data represent mean±SE values from 8 experiments.

FIGS. 3A and 3B depict western blots showing Der p1-induced activation of ERK1/2 and p38 MAP kinase in rabbit ASM tissues in the absence and presence of E-64. Tissue lysates were prepared at the indicated time points for immunoblotting using antibodies specific for phosphorylated and total ERK1/2 and p38 MAP kinase.

FIG. 4 is a graph showing a comparison of constrictor responses to ACh in rabbit ASM tissues exposed for 24 h to Der p1 in the absence and presence of SB202190 or U0126. Data represent mean±SE values.

FIG. 5 is a graph showing a comparison of relaxation responses to isoproterenol in rabbit ASM tissues exposed for 24 h to Der p1 in the absence and presence of the p38 MAPK inhibitor, SB202190 or the ERK1/2 inhibitor, U0126. Data represent mean±SE values.

FIG. 6 is a representative western blot and graph showing that inhibition of p38 MAP kinase with SB202190 enhances Der p1-induced ERK1/2 phosphorylation. Upper panel: representative Western blots depicting Der p1-induced temporal changes in phosphorylated ERK1/2 expression in control (non-pretreated) and SB202190-pretreated tissues. Lowerpanel: scanned densitometric results wherein the intensity of each phosphorylated ERK signal is normalized to its corresponding total ERK signal. Data represent mean±SE values from 3 experiments (*p<0.05).

FIG. 7 is a representative immunoblot showing that activation of p38 MAP kinase with anisomycin inhibits Der p1-induced enhanced phosphorylation of ERK1/2. Lysates for immunoblotting were prepared using control (unstimulated) ASM and tissues treated with aninomycin or Der p1 alone and in combination.

FIG. 8 is a graph showing a comparison of constrictor responses to ACh in rabbit ASM tissues exposed for 24 h to Der p 1, both in the absence and presence of pretreatment of the tissues with anisomycin (50 ng/ml). Data represent mean±SE values.

FIG. 9 is a graph showing a comparison of relaxation responses to isoproterenol in rabbit ASM tissues exposed for 24 h to Der p1, both in the absence and presence of pretreatment of the tissues with anisomycin. Data represent mean±SE values.

FIG. 10 is a schematic drawing of the signal transduction pathway leading to transcription factor activation and pro-asthmatic airway responsiveness.

FIG. 11 is a graph showing IgE-induced cytokine release from cultured human ASM cells.

FIGS. 12A and 12B are Western blots showing the induced MAP kinase activation as a function of phosphorylation of ERK1/2 and p38 on IgE-induced release of IL-6 from cultured human ASM cells (FIG. 12A). In contrast, ERK1/2 and p38 protein levels are not markedly different (FIG. 12B).

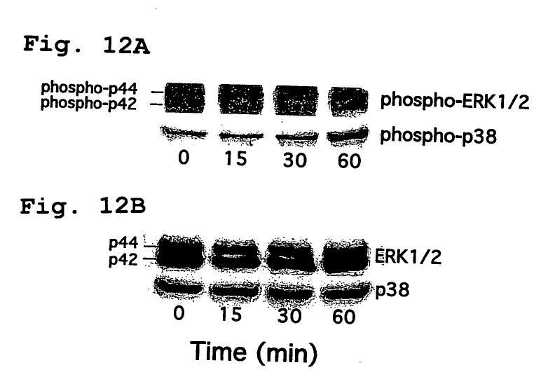

FIG. 13 is a graph showing the effect of inhibitors of MAP kinase on IgE-induced release of IL-6 from cultured ASM cells.

FIG. 14 is a graph showing the effect of inhibitors of MAP kinase on IgE-induced changes in ASM constrictor responses.

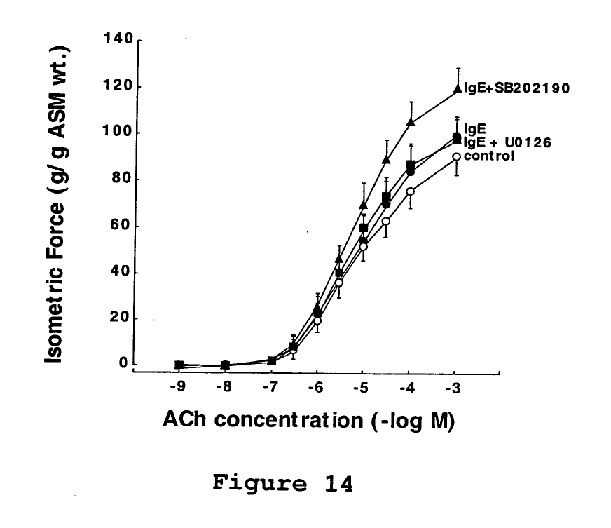

FIG. 15 is a graph showing the effect of inhibitors of MAP kinase on IgE-induced changes in ASM relaxant responses.

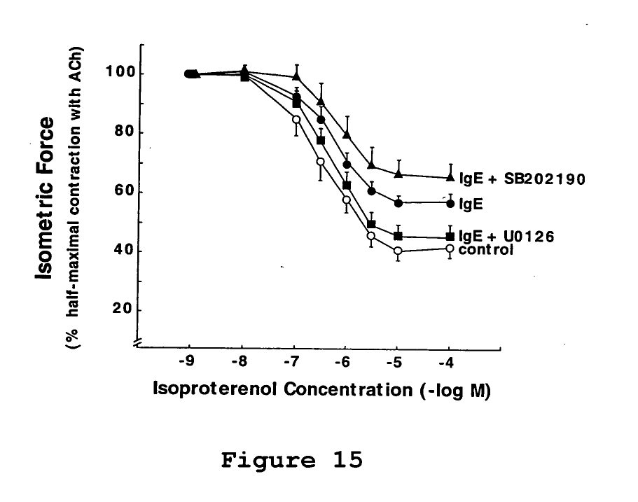

FIG. 16 is a Western blot showing the effects of MAP kinase inhibitors on expression of activated ERK1/2 in IgE-sensitized ASM cells.

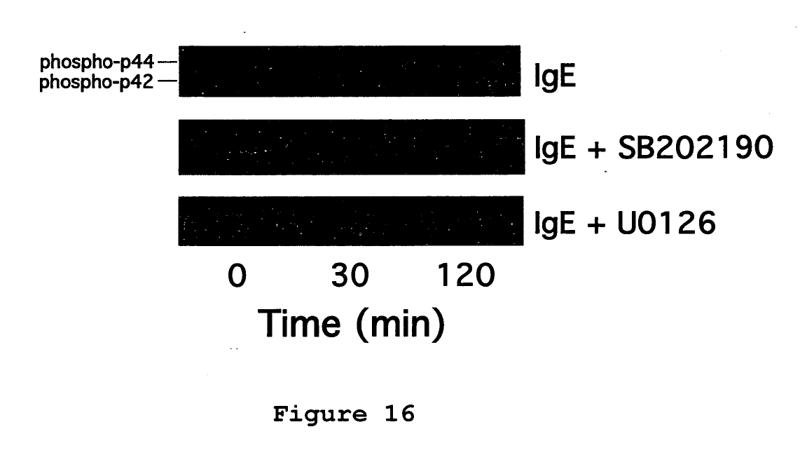

### DETAILED DESCRIPTION OF THE INVENTION

Pulmonary exposure to aeroallergens is a key risk factor for the development of allergic airway sensitization and asthma. Although the airway mucosal barrier is specialized in preventing the ingress of foreign proteins, many aeroallergens possess intrinsic protease activity that can disrupt the integrity of the airway epithelium (Stewart et al. (1996) Clin Exp Allergy 26:1020-44; Tomee et al. (1998) J Allergy Clin Immunol 102:75-85; King et al. (1998) J Immunol 161:3645-51; Kauffman et al. (2000) J Allergy Clin Immunol 105:1185-93; Asokananthan et al. (2002) J Immunol 169:4572-8).

In this respect, the cysteine protease activity of the dust mite allergen, Dermatophagoides pteronyssinus-1 (Der p1), was found to disrupt the epithelial cell tight junction proteins, ZO-1 and desmoplakin, thereby likely accounting for reported Der p1-induced changes in airway microvascular permeability (Wan et al. (1999) J Clin Invest 104:123-33; Herbert et al. (1995) Am J Respir Cell Mol Biol 12:369-78; Wan et al. (2000) Eur Respir J 15:1058-68). Moreover, Der p1 was found to induce proinflammatory cytokine and chemokine release from respiratory epithelial cells, (King et al., supra; Asokanathan et al., supra). Given this evidence, together with the important role attributed to dust mite allergens in the development of asthma (Sporik et al. (1990) N Engl J Med 323:502-7; Platts-Mills et al. (1992) J Allergy Clin Immunol 89: 1046-60; Peat et al. (1996) Am J Respir Crit Care Med 153:141-6), a disease characterized by altered airway constrictor and relaxant responsiveness, the present inventor examined whether dust mite allergen also exerts a direct action on airway smooth muscle (ASM) and, hence, potentially contributes to the induction of the pro-asthmatic phenotype of altered airway responsiveness.

The results obtained provide new evidence demonstrating that: 1) isolated rabbit ASM tissues directly exposed to Der p 1 exhibit altered agonist-mediated constrictor and relaxant responsiveness; 2) these induced pro-asthmatic-like changes in ASM function are regulated by concomitant activation of the extracellular signal-regulated kinase (ERK)-1/2 and p38 mitogen-activated protein (MAP) kinase signaling pathways in the Der p1-exposed ASM; and 3) the latter kinases exert opposing regulatory actions, wherein ERK1/2 activation mediates the Der p1-induced changes in ASM function, whereas p38 MAP kinase activation serves a homeostatic role by negatively regulating ERK1/2 activation, thereby limiting its pro-asthmatic effects.

Taken together, these findings indicate that exposure to house dust mite allergens can elicit pro-asthmatic-like changes in airway function that are attributed, at least in part, to a direct action of the allergen on ASM, the latter resulting in activation of MAP kinase-dependent signaling mechanisms that regulate ASM responsiveness.

We also present data which indicate that cytokine release and altered responsiveness exhibited by IgE-sensitized ASM is attributed to activation of ERK 1/2 and p38 MAP kinases.

The following definitions are provided to facilitate an understanding of the present invention.

The term “asthmatic state” as used herein, refers to the pro-asthmatic phenotype which is observed in airway smooth muscle cells. This phenotype is characterized by increased contraction and decreased relaxation of the airway tissue.

The phrase “treating asthma” refers to curing asthma, causing the symptoms of asthma to diminish, ablating or otherwise alleviating the disease.

The phrase “pro-asthmatic agent” refers to agents with stimulate the pro-asthmatic phenotype. Such agents include, without limitation, Der p1, toll-like receptor agonists (e.g., lipopolysaccharide), IgE, rhinovirus, respiratory syncytial virus and parainfluenza virus.

The phrase “anisomycin derivative” refers to chemical derivatives of anisomycin which retain the biological functions of the parent molecule. Anisomycin derivatives are described in U.S. Pat. No. 5,463,078 which is incorporated by reference herein and include, for example 4-O-dodecanoyl-3-O-carbamoyldeacetylanisomycin; 3-O-methylcarbarnoyl-deacetylanisomycin; 4-O-acetyl-3-O-carbamoyldeacetylanisomycin; 4-O-hexanoyl-3-O-carbamoyldeacetylanisomycin; 4-O-heptanoyl-3-O-carbamoyldeacetylanisomycin; 3-O-methoxymethyldeacetylanisomycin and 3-O-carbamoyldeacytylanisomycin.

The phrase “inflammatory response” refers to the cascade of events that occur when cells are challenged with antigen or allergen. Such responses include, without limitation, chemokine and cytokine release, stimulation and proliferation of macrophage cells, eosinophils and T lymphocytes, and activation of antibody producing B cells. In airway smooth muscle cells, an inflammatory response resulting from stimulation with allergen can adversely affect relaxation and constriction responses.

### Therapy

For therapeutic use, the compounds of the invention may be administered in any conventional dosage form in any conventional manner. Routes of administration include, but are not limited to, intravenous, intramuscular, subcutaneous, intrasynovial, infusion, sublingual, transdermal, oral, topical or inhalation via a nebulizer for example. The preferred modes of administration are via the systemic and inhalation routes.

The compounds of this invention may be administered alone or in combination with adjuvants that enhance stability of the inhibitors, facilitate administration of pharmaceutical compositions containing them in certain embodiments, provide increased dissolution or dispersion, increase inhibitory activity, provide adjunct therapy, and the like, including other active ingredients. Advantageously, such combination therapies utilize lower dosages of the conventional therapeutics, thus avoiding possible toxicity and adverse side effects incurred when those agents are used as monotherapies. Compounds of the invention may be physically combined with the conventional therapeutics for the treatment of asthma, (e.g., corticosteroids, sodium cromolyn, methylxanthines, leukotriene modifiers and rapid relief medications that counteract bronchospasm, e.g., beta-adrenergic agents) or other adjuvants into a single pharmaceutical composition. Advantageously, the compounds may then be administered together in a single dosage form. In some embodiments, the pharmaceutical compositions comprising such combinations of compounds contain at least about 15%, but more preferably at least about 20%, of a compound of the invention (w/w) or a combination thereof. Alternatively, the compounds may be administered separately (either serially or in parallel). Separate dosing allows for greater flexibility in the dosing regime.

As mentioned above, dosage forms of the compounds of this invention include pharmaceutically acceptable carriers and adjuvants known to those of ordinary skill in the art. These carriers and adjuvants include, for example, ion exchangers, alumina, aluminum stearate, lecithin, serum proteins, buffer substances, water, salts or electrolytes and cellulose-based substances. Preferred dosage forms include, tablet, capsule, caplet, liquid, solution, suspension, emulsion, lozenges, syrup, reconstitutable powder, granule, suppository and transdermal patch. Methods for preparing such dosage forms are known (see, for example, H. C. Ansel and N. G. Popovish, Pharmaceutical Dosage Forms and Drug Delivery Systems, 5th ed., Lea and Febiger (1990)). Dosage levels and requirements are well-recognized in the art and may be selected by those of ordinary skill in the art from available methods and techniques suitable for a particular patient. In some embodiments, dosage levels range from about 10-1000 mg/dose for a 70 kg patient. Although one dose per day may be sufficient, up to 5 doses per day may be given. For oral doses, up to 2000 mg/day may be required. As the skilled artisan will appreciate, lower or higher doses may be required depending on particular factors. For instance, specific dosage and treatment regimens will depend on factors such as the patient's general health profile, the severity and course of the patient's disorder or disposition thereto, the patient's age, and the judgment of the treating physician.

### RATIONAL DRUG SCREENING AND DESIGN

The discoveries made in the present invention provide the basis for screening methods which are amenable for identifying specific agonists of p38 MAPK which have efficacy for the treatment of asthma. Such screening methods may take many forms.

For example, cultured respiratory epithelial cells may be obtained, challenged with allergen and alterations in conventional inflammatory responses in the presence and absence of a test compound determined. Such alterations can include, without limitation, altered levels of phosphorylation of p38 MAP kinase and ERK1/2, modulation of amounts of inflammatory cytokine (e.g., IL-6) and chemokine release. When airway smooth muscle tissue or cells are employed modulation of airway constriction and relaxation may be assessed. The skilled person is well aware of the many methods available to detect phosphorylation states/levels in proteins of interest. See for example, the western blotting methods employed in Examples 1 and 2. Airway smooth muscle cell tissue relaxation and constriction responses may also be measured as described herein.

It is known that APCs, including dendritic cells, monocytes, macrophages and B cells, possess functional receptors for numerous molecules that modulate the immune response. Accordingly, co-culture of dendritic cells with antigen presenting cells provides a suitable system for screening test compound for agonistic activity against p38 MAP kinase. See Romani et al. (1989) J. Exp. Med. 169:1169. Optionally, cytokine release in the presence and absence of such test compounds can be assessed. For methods for the production and culture of dendritic cells, see de Saint-Vis et al., (1998) J of Immunol. 160:1666-1676.

In another approach, test compounds can be screened in vivo in whole animal models of asthma. Animals (typically mice) are sensitized to exogenous allergen (usually, ovalbumin (OVA) by daily intra-peritoneal injection of 20-30 μg of OVA together with 3-4 mg of aluminium hydroxide for 14 days. One to two weeks thereafter, the animals are challenged by aerosol inhalation of OVA (usually 1% in saline) for 15-20 minutes once per day for 2-3 days. In vivo airway responsiveness to a bronchoconstrictor agonist (typically, methacholine) is subsequently assessed. Additionally, lung fluid samples are obtained by bronchoalveolar lavage of the lungs to assess for the presence of different types of inflammatory cells, as well as for the presence of cytokines, chemokines, and other proinflammatory mediators. Finally, the lungs are histologically examined for the presence of inflammation and markers of inflammatory proteins. Accordingly, the model may be employed to assess phosphorylation states of p38 and/or ERK1/2 in biopsied lung tissue in the presence and absence of administration of the test compound (Duan et al., (2004) J. of Immun. 172:7053-7059; Choudhury et al. (2002) J. of Immun. 169:5955-5961).

Agents identified as putative agonists of p38 MAP kinase can include small molecules, antibiotics peptides, and the like. Small molecules in combinatorial chemical libraries can be screened as set forth above. Once candidate molecules have been identified, such molecules may be further optimized to enhance binding and activation of p38 MAPK.

The following examples are provided to facilitate the practice of the present invention. They are not intended to limit the invention in any way.

### EXAMPLE I

House dust mite (HDM) allergen exposure is a key risk factor for the development of allergic asthma. Beyond provoking immune cell-mediated allergic responses, HDM allergens were recently shown to exert direct effects on airway structural cells secondary to their intrinsic protease activities. In the present invention, we have tested the hypothesis that HDM allergen exposure produces changes in airway responsiveness due to a direct effect on airway smooth muscle (ASM). Isolated rabbit ASM tissues were exposed to the HDM allergen, Der p1, and induced changes in ASM responsiveness and activation of mitogen-activated protein (MAP) kinase signaling pathways were examined under different experimental conditions. The results demonstrated that: 1) Der p1 exposure elicited enhanced constrictor responses and impaired relaxation responses in the ASM tissues; 2) these pro-asthmatic-like effects of Der p1 were attributed to its intrinsic cysteine protease activity; and 3) the induced changes in ASM responsiveness were associated with activation of both the ERK1/2 and p38 MAP kinase signaling pathways. Additionally, specific blockade of ERK1/2 signaling was found to prevent the Der p1-induced changes in ASM responsiveness, whereas inhibition of p38 MAP kinase signaling enhanced the pro-asthmatic-like action of Der p1, the latter effect due to augmented activation of ERK1/2.

These findings are the first to demonstrate that the dust mite allergen, Der p1, elicits changes in ASM responsiveness that are associated with activation of MAP kinase signaling, wherein pro-asthmatic effects induced by Der p1 are attributed to activation of ERK1/2 whereas co-activation of p38 MAP kinase exerts a homeostatic action by negatively regulating ERK1/2 signaling and hence, its pro-asthmatic action.

The materials and methods set forth below are provided to facilitate the practice of the present invention.

Animals

Thirty-four adult New Zealand White rabbits were used in this study, which was approved by the Biosafety and Animal Research Committee of the Joseph Stokes Research Institute at Children's Hospital of Philadelphia. The animals had no signs of respiratory disease for several weeks before the study, and their care and use were in accordance with the “Guide for the Care and Use of Laboratory Animals” prepared by the Institute of Laboratory Animal Resources, National Research Council.

Preparation and Der p1 Treatment of Rabbit ASM Tissues for Example I

Following general anesthesia with xylazine (10 mg/kg) and ketamine (50 mg/kg), rabbits were sacrificed with an overdose of pentobarbital (125 mg/kg). As described previously (Grunstein et al. (2001) Am J Physiol Lung Cell Mol Physiol 280:L229-L238), the tracheae were removed via open thoracotomy, the loose connective tissue and epithelium were carefully scraped and removed, and the tracheae were divided into ring segments. Each alternate ring was incubated for 24 hours at room temperature in the presence of either vehicle (saline) alone (control), vehicle containing cysteine (5 mM), or varying concentrations of affinity purified Der p1 allergen (0.1, 0.5, 1.0, 5.0 μg/ml) both in the absence and presence of cysteine, as addition of the latter is required to restore the cysteine protease activity of the purified Der p1. In parallel experiments, 30 min prior to incubation in control or Der p1-containing medium, ASM segments were treated with either the specific cysteine protease inhibitor, E-64 (trans-epoxysuccinyl-L-leucyl-amido (4-guanidino)-butane; 1 μM), the serine protease inhibitor, leupeptin (1 μM), the MEK1/2-specific inhibitors, U0126 (5 μM) or PD98059 (10 μM), or the specific p38 MAP kinase inhibitors, SB202190 (10 μM) or SB203580 (10 μM). Finally, in separate studies, 1 hour before incubation in control or Der p1-containing medium, ASM tissues were treated with the potent activator of p38 MAP kinase, anisomycin, using a concentration (i.e., 50 ng/ml) that is significantly less than that required to prevent the known protein translational inhibitory effect of anisomycin (Hazzalin et al. (1998) Mol Cell Biol 18: 1844-54).

Human ASM cell culture

Human airway smooth muscle cells were purchased from BioWhittaker, Inc., (Walkersville, Mich.) The cells were cultured in 100 mm dishes in Ham's F 12 nutrient mixture (Invitrogen, Carlsbad Calif.) supplemented with 10% FBS (Hyclone, Logan, Utah) under 5% CO₂ environment. After attaining 90% confluence in cell culture, the cells were used for the studies described in Example 2.

Sensitization of Cultured Human ASM Cells with IgE Immune Complexes

Preparations of IgE immune complexes containing human IgE and mouse monoclonal IgG2B (anti-IgE) (Biodesign International, Saco, Me.) at a 15/5 μg ratio were added to cells 24 hr after serum deprivation. The cells were harvested at the indicated time points for immunoblotting and media were collected for ELISA analysis as described below.

Preparation and Treatment of Rabbit ASM Tissues for Example 2

New Zealand White rabbits were sacrificed by an overdose of sodium pentobarbital. The tracheae were removed, cleaned of loose connective tissue and epithelium, and divided into ring segments. Each alternate ring was incubated for 24 hr with either vehicle alone or IgE immune complexes. In parallel experiments, 1 hr prior to incubation, ASM segments were treated with the MEK 1/2 inhibitor, U 0126 (5 μM), or the p38 MAP kinase inhibitor, SB-202190 (10 μM).

Pharmacodynamic Studies of ASM Responsiveness

Following incubation, the ASM segments were placed in organ baths containing modified Krebs-Ringer solution aerated with 5% CO₂ in oxygen (pH: 7.35-7.40), and the tissues were attached to force transducers from which isometric tension was continuously monitored on a multichannel recorder, as previously described (Hirst et al. (2002) Am J Respir Crit Care Med 165:1161-71). Cholinergic contractility was then assessed in the ASM segments by cumulative administration of acetylcholine (ACh; 10⁻¹⁰ to 10⁻⁴ M). Thereafter, the tissues were repeatedly rinsed with fresh buffer, and subsequent relaxation responses to isoproterenol (10⁻¹⁰-10⁻⁴ M) were generated after the tissues were half-maximally contracted with ACh. The constrictor dose-response curves to ACh were analyzed in terms of each tissue's maximal isometric contractile force (Tmax) to the agonist, and the relaxation responses to isoproterenol were assessed in terms of % maximal relaxation (Rmax) from the initial level of induced cholinergic contraction, and sensitivity to the relaxing agent was determined as the corresponding pD50 value (i.e., geometric mean ED50 value) associated with 50% of Rmax.

Immunoblot Analysis of MAP Kinase Activation

Protein levels of phosporylated and total ERK1/2 and p38 MAP kinase were assessed by Western blot analysis of whole cell lysates isolated from rabbit ASM tissues following treatment with either vehicle alone or cysteine-activated Der p1 (1 μg/ml), both in the absence and presence of E-64, SB202190, or anisomycin. The tissues were homogenized and proteins extracted in a buffer containing 50 mM Tris-HC 1,150 mM NaCl, 1 mM EDTA (pH 7.4) with 1 mM phenylmethylsulfonyl fluoride, 5 μg/mL aprotinin, and 5 μg/mL leupeptin. Following removal of insoluble debris by centrifugation, gel-loading buffer was added to the supernatants, and extracts were then loaded (using 30 μg total protein/sample) on a 10% SDS-PAGE gel for immunoblotting after transferring to a nitrocellulose membrane. The membranes were blocked with 5% nonfat dry milk and then incubated overnight with monoclonal mouse anti-human phosphotyrosine-threonine (Thr180/Tyr182)-p38 MAP kinase and p38 MAP kinase, and anti-human phosphotyrosine-threonine (Thr202/Tyr204)-p44/42 (ERK1/2) and p44/42 primary antibodies. The kinase levels were detected using enhanced chemiluminescence after a 1-hour incubation with a 1:1,000 dilution of horseradish peroxidase (HRP)-conjugated goat-anti-mouse secondary antibody, followed by exposure to autoradiography film. Protein band intensities were quantified using Alphalmager, and the data normalized relative to those obtained in untreated ASM samples.

Measurement of Intracellular cAMP Accumulation

Intracellular cAMP levels were assayed in control (untreated) and Der p1-treated ASM preparations under baseline (unstimulated) conditions and at 10 min following administration of isoproterenol (0.1-10 μM). Following stimulation with isoproterenol, the tissues were homogenized and the cAMP levels were measured using the Form a A cyclic AMP “PLUS” EIA kit (Biomol Inc., Plymouth Meeting, Pa.) following manufacturer's protocol.

Detection of IL-6 in the ASM Culture Media

IL 6 accumulation in the ASM cell culture media was detected using an ELISA kit for IL 6 purchased from Research Diagnostics Inc. (Flanders, N.J.). Culture media was diluted 1:50 into a sample dilution buffer supplied in the kit. The experiments were carried out according to the manufacturer's protocol.

Statistical Analysis

The results are expressed as mean±SE values. Comparisons between groups were made using the Student's t-test (2-tailed) or ANOVA with Tukey's post-test analysis, where appropriate. A probability of <0.05 was considered statistically significant. Statistical analyses were conducted using the Prism computer program by GraphPad Software Inc. (San Diego, Calif.).

Reagents

All chemicals were purchased from Sigma (St. Louis, Mo.) unless otherwise indicated. Der p1 was obtained from Indoor Biotechnologies (Charlottesville, Va.). The MEK and p38 MAP kinase inhibitors were purchased from EMD Biosciences Inc. (San Diego, Calif.). The primary antibodies for Western blotting were obtained form Santa Cruz Biotechnology, Inc. (Santa Cruz, Calif.), and the ECL reagents and goat-anti-mouse secondary antibody were from Amersham Pharmacia Biotech. (Piscataway, N.J.). All drug concentrations are expressed as final both concentrations. Isoproterenol and acetylcholine were made fresh for each experiment, and were dissolved in normal saline to prepare 10-3 M stock solutions.

### Results

Effects of Der p1 on Rabbit ASM Responsiveness

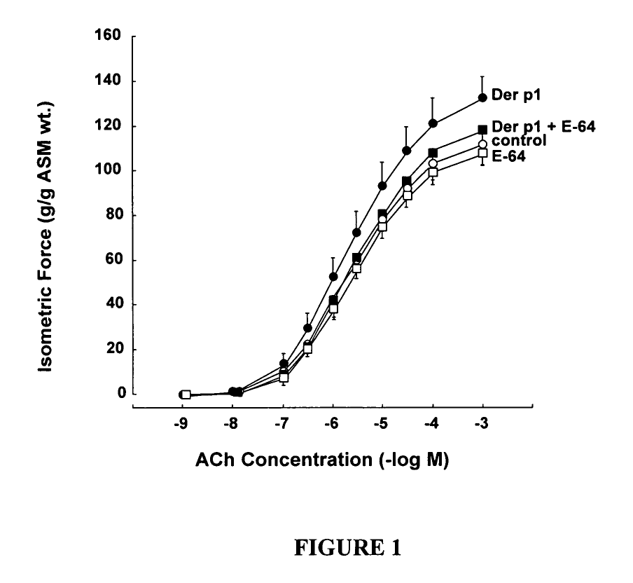

To assess the effects of Der p1 on ASM responsiveness, agonist-mediated constrictor and relaxation responses were compared in control and Der p1-exposed isolated rabbit ASM segments, both in the absence and presence of E-64 (1 μM). As shown in FIG. 1, relative to control tissues, ASM segments exposed to a maximally effective dose of Der p1 (i.e., 1 μg/ml) exhibited increased constrictor responses to exogenously administered ACh. Accordingly, relative to the mean±SE maximal constrictor response (Tmax) obtained in control ASM, which amounted to 111.4±7.4 g/g ASM wt., the mean Tmax response was significantly increased at 133.7±8.4 g/g ASM wt. in the Der p1-treated ASM tissues (p<0.05). This increased constrictor responsiveness to ACh was largely abrogated in Der p1-exposed ASM that were pretreated with E-64, whereas the latter had no effect on the constrictor responses to ACh in tissues that were not exposed to Der p1.

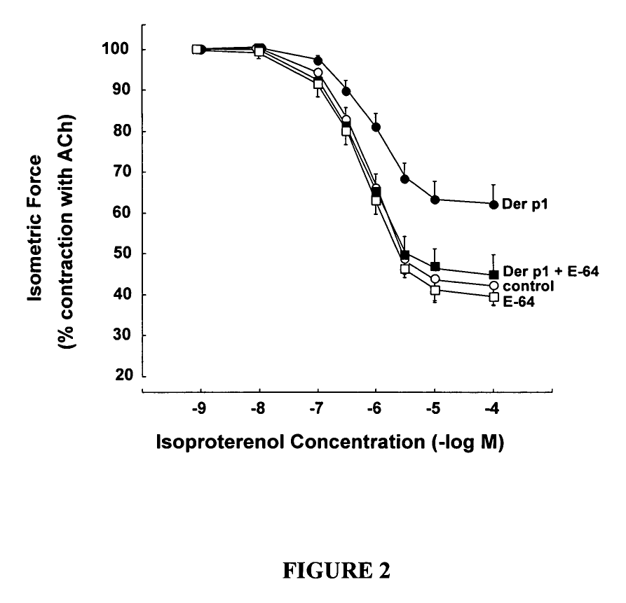

Under the same treatment conditions, during subsequent sustained half-maximal contraction with ACh, administration of the β-adrenoceptor agonist, isoproterenol, produced cumulative dose-dependent relaxation of the pre-contracted ASM segments. As depicted in FIG. 2, relative to control ASM, the relaxation responses to isoproterenol were significantly attenuated in the Der p1-exposed ASM. Accordingly, the mean±SE maximal relaxation (Rmax) values obtained in the Der p1-treated vs. control ASM tissues amounted to 37.9±4.9 vs. 57.4±4.0%, respectively (p<0.01), and the corresponding sensitivities to isoproterenol (i.e., pD50 values) averaged 5.99±0.04 and 6.22±0.03-log M, respectively (p<0.05). This impaired relaxation responsiveness to isoproterenol was prevented in Der p 1-exposed tissues that were pretreated with E-64, whereas the latter had no effect in tissues that were not exposed to Der p1.

In relation to the above observations, it should be noted that, in comparable experiments conducted on ASM tissues exposed to Der p1 that were not pre-activated with cysteine (i.e., required to restore the cysteine protease activity of the purified allergen), we found no effect of the inactivated Der p1 on the tissues' constrictor or relaxant responsiveness (data not shown). Moreover, contrasting the above effects of cysteine protease inhibition with E-64, we found that the induced changes in constrictor and relaxant responsiveness elicited in ASM exposed to cysteine-activated Der p1 were unaffected by pretreating the tissues with the serine protease inhibitor, leupeptin (1 μM) (data not shown). Finally, we also observed that relative to control (untreated) ASM, tissues exposed to Der p1 exhibit reduced isoproterenol-induced cAMP accumulation (data not shown).

MAP Kinase Activation in Der p1-Exposed ASM

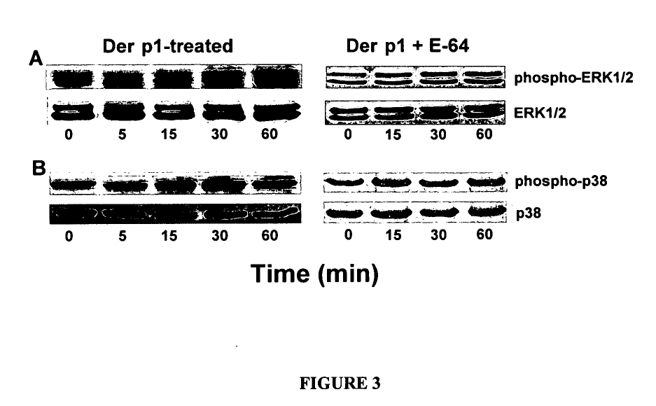

Previous studies have implicated activation of MAP kinases in mediating the changes in airway responsiveness in animal models of allergic asthma (Tsang et al., (1998) Br J Pharmacol 125:61-8; Duan et al., (2004) J Immunol 172:7053-9), and the induced release of various cytokines and chemokines by stimulated ASM cells. Given this evidence, we examined the role of MAP kinase activation in regulating the above Der p1-induced changes in ASM responsiveness. MAP kinase activation was assessed by Western blot analysis, using antibodies specific for the phosphorylated forms of ERK1/2 and p38 MAPK, as well as antibodies specific for total ERK and p38 proteins. As exemplified in FIG. 3A (left panel), enhanced expression of phosphorylated ERK1/2 was detected as early as 15 min, with progressively increased expression for up to 60 min following Der p1 exposure. Enhanced expression of phosphorylated p38 MAP kinase appeared within 5 min, and was transiently increased for up to 30 min thereafter, but was distinctly reduced by 60 min following Der p1 exposure (FIG. 3B; left panel). Based on densitometric analysis of the results obtained in 4 experiments, the magnitudes of maximal enhanced expression of phosphorylated ERK1/2 and p38 MAP kinase detected in Der p1-exposed tissues averaged 5.8- and 3.2-fold above the corresponding basal (pretreatment) levels, respectively. Contrasting these observations, neither the levels of phosphorylated ERK1/2 nor p38 MAP kinase were appreciably altered in response to Der p1 exposure in tissues that were pretreated with E-64 (FIGS. 3A and 3B: right panels), Moreover, neither total ERK nor p38 MAP kinase protein levels were systematically affected by Der p1 exposure in either the absence or presence of E-64.

Role of MAP Kinases in Regulating Der p1-Induced Changes in ASM Responsiveness

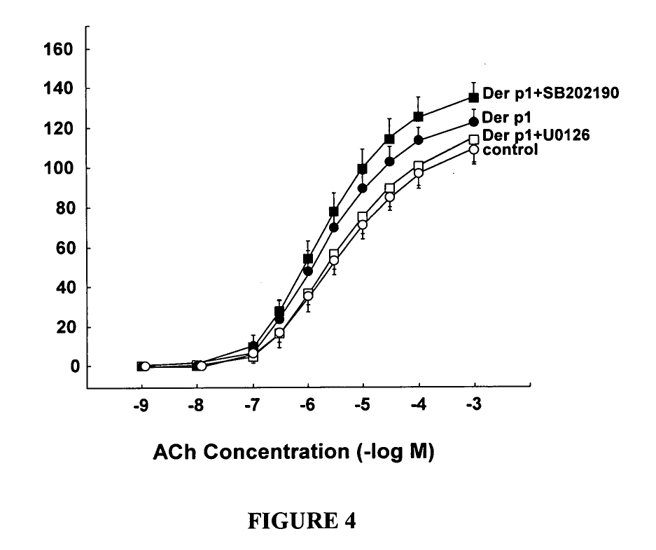

In light of the above results, we next investigated the potential regulatory role(s) of MAP kinase activation by assessing the effects of selective inhibitors of the MEK-ERK1/2 and p38 MAP kinase pathways on Der p1-induced changes in ASM responsiveness. As shown in FIG. 4, relative to control tissues, ASM exposed to Der p1 exhibited enhanced maximal constrictor responses to ACh, providing a mean±SE Tmax value of 123.8±6.5 g/g ASM wt., relative to the value of 109.9±8.7 g/g ASM wt. obtained in control ASM (p<0.05). Interestingly, this enhanced constrictor responsiveness was further augmented in Der p1-exposed tissues that were pretreated with the specific p38 MAP kinase inhibitor, SB202190 (10 μM), yielding an average Tmax value (i.e., 136.2±6.2 g/g ASM wt.) that was significantly greater (p<0.05) than that obtained in the Der p1-exposed tissues which did not receive the inhibitor. In marked contrast to the latter effect of p38 MAP kinase inhibition, the changes in ASM constrictor responsiveness to ACh were completely abrogated in Der p1-exposed tissues that were pretreated with the specific MEK1/2 inhibitor, U0126 (5 μM), providing Tmax values that were similar to those generated in control ASM segments.

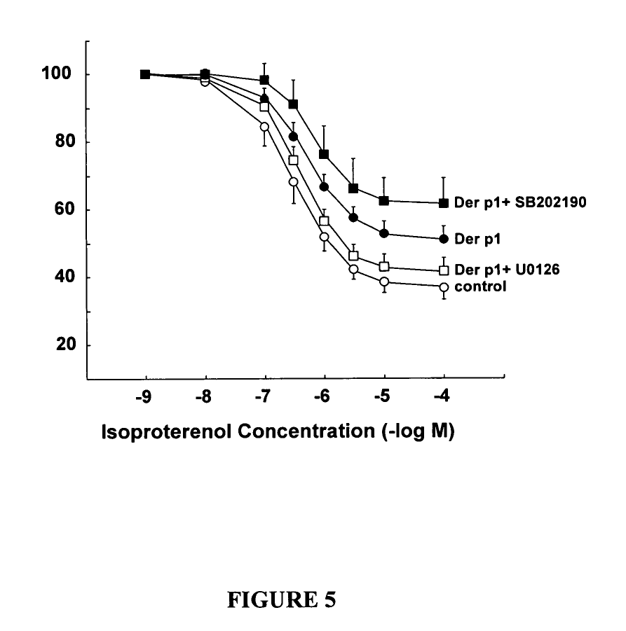

In parallel to the above observations, relative to control tissues, the relaxation responses to isoproterenol were significantly attenuated in Der p1-exposed ASM tissues (FIG. 5), providing Rmax values that averaged 48.8±4.7%, as compared to the mean Rmax of 62.6±4.6% obtained in control tissues (p<0.05). This attenuated relaxant responsiveness was further significantly impaired (p<0.05) in Der p1-exposed tissues that were pretreated with SB202190, wherein the mean Rmax amounted to 37.6±7.1% (p<0.01). By comparison, the reduced relaxation responsiveness to isoproterenol was largely ablated in Der p1-exposed ASM tissues that were pretreated with U0126, providing Rmax values that were not significantly different from those obtained in control ASM segments.

As with the above inhibitors, in separate experiments we found qualitatively comparable opposing effects of p38 and ERK1/2 inhibition on ASM responsiveness in Der p1-exposed tissues that were pretreated with other selective MAP kinase inhibitors, including the p38 MAP kinase inhibitor, SB203580, and the MEK-ERK1/2 inhibitor, PD98059 (data not shown). Moreover, contrasting their effects in Der p1-exposed tissues, neither of these inhibitors produced a significant change in ASM constrictor or relaxant responsiveness when administered to tissues that were not exposed to Der p1 (data not shown).

Modulatory Effect of p38 MAP Kinase on ERK1/2 Activation in Der p1-Exposed ASM

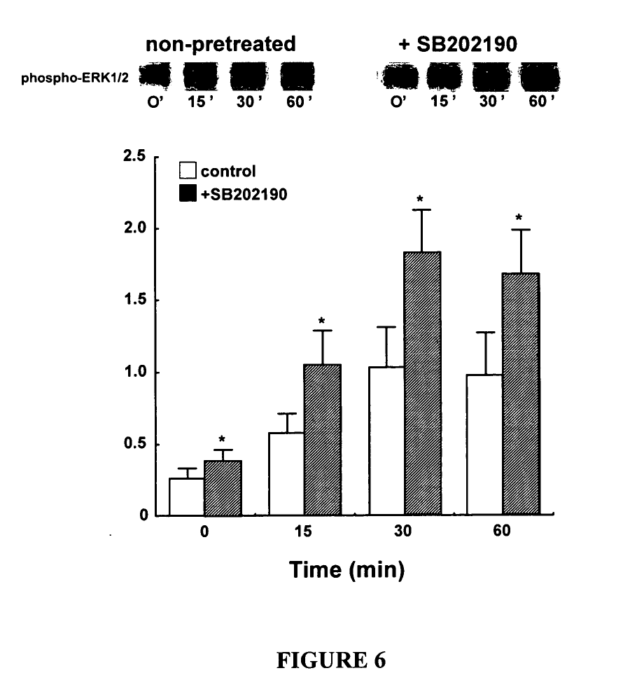

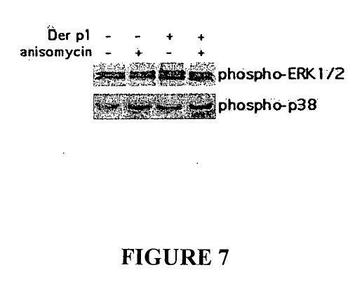

The above observations suggested that the Der p1-induced changes in ASM responsiveness were mediated by activation of the MEK-ERK1/2 signaling pathway, whereas concomitant activation of p38 MAP kinase exerted an opposing regulatory effect. This consideration appeared consistent with the findings in recent reports that demonstrated a negative regulatory interaction between the ERK1/2 and p38 MAP kinase signaling pathways, wherein p38 MAP kinase activation was found to inhibit ERK1/2 signaling (Zhang et al., (2001) J Biol Chem 276:6905-8), and, conversely, inhibition of p38 MAP kinase was shown to activate ERK1/2 (Singh et al., (1999) J Biol Chem 274:19593-600; Rosenberger et al., (1999) Oncogene 18:3626-32; Birkenkamp et al., (2000) Brit J Pharmacol 131:99-107). To address the latter mechanism herein, we examined the separate effects of inhibition and stimulation of p38 MAP kinase on Der p1-induced activation of ERK1/2. As exemplified by the Western blots depicted in the top panel of FIG. 6, relative to tissues that were not pretreated with the p38 MAP kinase inhibitor, Der p1-exposed ASM tissues that were pretreated with SB202190 displayed distinctly enhanced time-related expression of phosphorylated ERK1/2. Total ERK protein levels were not systematically affected by SB202190 and, hence, the time-specific ratios of the levels of phosphorylated to total ERK protein were significantly increased in the Der p1-exposed tissues that were pretreated with SB202190 (FIG. 6; bottom panel). These observations implicating a negative regulatory effect of p38 MAP kinase on Der p1-induced ERK1/2 activation were further substantiated in separate experiments wherein the effects of anisomycin, a potent activator of p38 MAP kinase, were compared in control and Der p1-exposed ASM tissues. As exemplified by the immunoblots in FIG. 7, relative to control (unstimulated) ASM, tissues exposed for 30 min to either anisomycin (50 ng/ml) or Der p1 alone, or both in combination, exhibited increased expression of phosphorylated p38 MAP kinase. Correspondingly, treatment with anisomycin had essentially no effect on phosphorylated ERK1/2 expression in unstimulated ASM; however, the induced enhanced expression of phosphorylated ERK1/2 exhibited by Der p1-exposed ASM was largely abrogated when Der p 1-exposed tissues were co-treated with anisomycin. Of note, neither the levels of total p38 MAP kinase nor ERK 1/2 protein were systematically altered by these different treatment conditions (data not shown). Thus, taken together, these observations support the above concept that activation of p38 MAP kinase in Der p1-exposed ASM serves, at least in part, to limit the magnitude of Der p1-stimulated co-activation of ERK1/2.

Effect of p38 MAP Kinase Activation on Der p1-Induced Changes in ASM Responsiveness

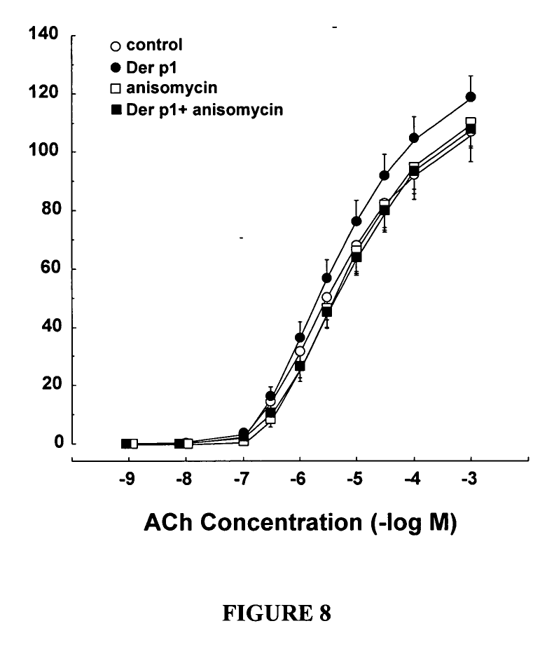

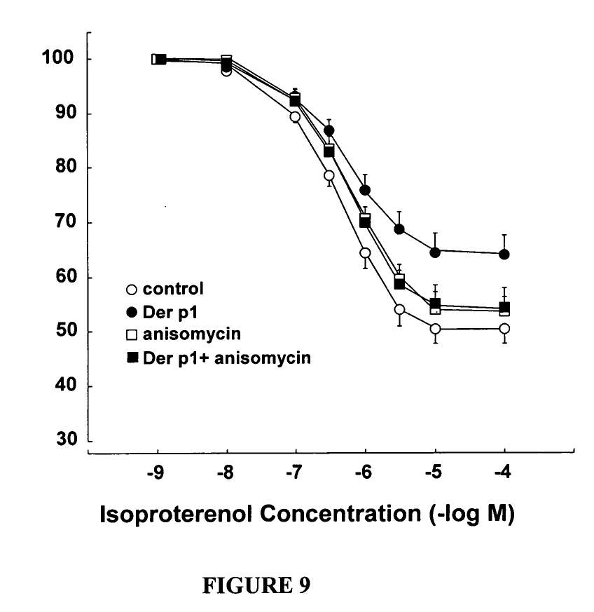

Given the above results, we next examined whether the negative regulatory effect of p38 MAP kinase on ERK1/2 activation is reflected by a protective action of p38 MAP kinase activation on Der p1-induced changes in ASM responsiveness. Accordingly, constrictor and relaxation responses were compared in control and Der p1-exposed tissues both in the absence and presence of pretreatment of the tissues with anisomycin (50 ng/ml). As depicted in FIG. 8, relative to control ASM segments (open circles), Der p1-exposed tissues exhibited increased constrictor responses to ACh (filled circles), wherein the mean±SE Tmax value amounted to 119.0±8.2 g/g ASM wt., compared to the corresponding value of 107.4±6.6 g/g ASM wt. obtained in the control ASM (p<0.05). This enhanced constrictor responsiveness to ACh was completely abrogated in Der p1-exposed ASM tissues that were pretreated with anisomycin (filled squares), whereas the latter had no effect in control (vehicle-exposed) ASM segments (open squares). Additionally, the impaired relaxation responsiveness to isoproterenol exhibited in Der p1-exposed ASM segments was also largely prevented in tissues that were pretreated with anisomycin, whereas the latter had no significant effect in control ASM (FIG. 9). Furthermore, in relation to these observations, given that anisomycin is known to activate both p38 MAP kinase and the stress-activated protein kinase, c-Jun NH2-terminal kinase (JNK), in extended experiments we examined whether the effects of anisomycin on Der p1-induced changes in ASM responsiveness were attributed, at least in part, to co-activation of JNK. The results demonstrated that the protective effect of anisomycin on Der p1-induced changes in ASM responsiveness was not appreciably affected in Der p1-exposed tissues that were co-pretreated with the JNK inhibitor, SP600125 (1 μM) (data not shown).

### Discussion

To our knowledge, the results of the present study are the first to demonstrate that the ubiquitous aeroallergen, Der p1, can directly exert pro-asthmatic-like effects on ASM function that are characterized by enhanced agonist-mediated ASM contractility and impaired β-adrenoceptor-mediated ASM relaxation. Our observed actions of purified Der p1 on ASM function were attributed to its intrinsic cysteine protease activity since the induced changes in ASM responsiveness were largely prevented in Der p1-exposed ASM cells and tissues that were pretreated with the cysteine protease inhibitor, E-64. Additionally, the specificity of action of Der p1 as a cysteine protease was substantiated by the finding that ASM responsiveness was unaltered in tissues that were exposed to Der p1 which was not pre-activated with cysteine, the latter required to restore the cysteine protease activity of the purified allergen. In relation to these observations, it is relevant to note that the reported cellular responses to the proteolytic activities of Der p1 and other dust mite allergens have been attributed to activation of one or more members of a group cell surface receptors comprising the protease-activating receptor (PAR) family. Cell surface expression of PARs has been identified in the airways of different animal species including humans (Knight et al., (2001) J Allergy Clin Immunol 108:797-803; D'Andrea et al., (1998) J Histochem Cytochem 46:157-64), and, among the PARs, particular attention has been given to PAR-2, given that this receptor subtype has been implicated in mediating the evoked release of cytokines from respiratory epithelial cells exposed to Der p1 and other Der p allergens, as well as to cockroach extract, the latter associated with ERK activation (Page et al., (2003) J Allergy Clin Immunol 112: 1112-8). Additionally, PAR-2 activation has been shown to induce the release of cyclooxygenase products from epithelial and ASM cells, and to provoke enhanced contractility in isolated human bronchial preparations. These earlier findings suggest that the observed effects of Der p 1 in ASM were likely attributed to PAR-2 activation, although the involvement of PAR-2 and/or other PARs in mediating to our observed effects of Der p 1 on ASM responsiveness remains to be systematically investigated.

Recent evidence implicates a crucial role for activation of the MAP kinase signaling cascade in regulating cytokine synthesis by stimulated ASM cells, most notably including the ERK1/2 and p38 MAP kinase pathways. Our present observations extend these earlier findings by demonstrating that the induced changes in ASM responsiveness in Der p1-exposed ASM are also associated with activation of both the MEK-ERK1/2 and p38 MAP kinase pathways (FIG. 3). Moreover, in examining the roles of these signaling pathways, we found that pretreatment with a MEK1/2 inhibitor, U0126 or PD98059, largely prevented the Der p1-induced changes in ASM constrictor and relaxant responsiveness (FIGS. 4 and 5). These observations fundamentally agree with those reported in recent studies that have demonstrated an important role for ERK1/2 activation in mediating the in vitro changes in bronchial contractility observed in ovalbumin (OVA) sensitized guinea pigs, as well the in vivo airway hyperresponsiveness seen in OVA-sensitized mice. In the latter study, treatment with the MEK-ERK 1/2 inhibitor, U0126, was found to significantly inhibit the airway hyperresponsiveness and elevated intrapulmonary eosinophil counts and cytokine levels in OVA-sensitized mice.

Contrasting the role of ERK activation, our present observations demonstrated that co-activation of p38 MAP kinase exerts a homeostatic (i.e., protective) action in Der p1-exposed ASM by attenuating the pro-asthmatic effects mediated by ERK1/2 activation. Accordingly, we found that, along with induced potentiation of Der p1-induced changes in ASM responsiveness (FIGS. 4 and 5), inhibition of p38 MAP kinase elicited enhanced expression of phosphorylated ERK1/2 in Der p1-exposed ASM (FIG. 6). The latter finding concurs with earlier observations made in other cell systems wherein inhibition of p38 MAP kinase was also found to enhance ERK1/2 phosphorylation and, thereby, augment ERK1/2-mediated cellular responses. These previous findings, together with our present observations, are consistent with the concept that p38 MAP kinase negatively regulates the ERK1/2 signaling pathway. Supporting this concept, a direct one-way cross-talk between p38 MAP kinase and ERK1/2 was recently demonstrated, wherein phosphorylated p38 was found to couple with ERK1/2 and, thereby, sterically block ERK1/2 phosphorylation by MEK1/2, 22 and possibly also act via a protein kinase that lies upstream of MEK1/2. Our results herein provide evidence that concurs with this cross-talk mechanism, given the observations that activation of p38 MAP kinase with anisomycin largely prevented both the Der p1-induced enhanced phosphorylation of ERK1/2 (FIG. 7) and changes in ASM responsiveness (FIGS. 8 and 9), whereas anisomycin had no effect in control ASM preparations. Our latter findings concur with those previously reported using HL-60 leukemia cells which showed that anisomycin inhibits stimulated ERK activity while having no effect on basal ERK activity in unstimulated cells.

In conclusion, the present study investigated the role and mechanism of action of the dust mite allergen, Der p1, in regulating ASM responsiveness. The results provided new evidence demonstrating that: 1) Der p1 exposure provokes pro-asthmatic-like changes in constrictor and relaxant responsiveness in isolated rabbit ASM tissues; 2) these effects of Der p1 are attributed to its intrinsic cysteine protease activity, which elicits activation of both the ERK1/2 and p38 MAP kinase intracellular signaling pathways; 3) activation of ERK1/2 is responsible for mediating the Der p1-induced changes in ASM responsiveness; and 4) contrasting this effect, co-activation of p38 MAP kinase serves to homeostatically limit the magnitude of Der p 1-induced changes in ASM responsiveness by down-regulating ERK1/2 activity and, hence, its pro-asthmatic-like effects. Collectively, these observations provide new information demonstrating that exposure to the dust mite allergen, Der p1, can directly elicit pro-asthmatic-like changes in ASM function and, accordingly, the findings support the novel concept that ASM may play an important innate role in contributing to the acquisition of dust mite allergen-induced airway sensitization and asthma.

### EXAMPLE 2

### Role of Map Kinases in Regulating IgE-Induced Cytokine Protection by Human Airway Smooth Muscle Cells

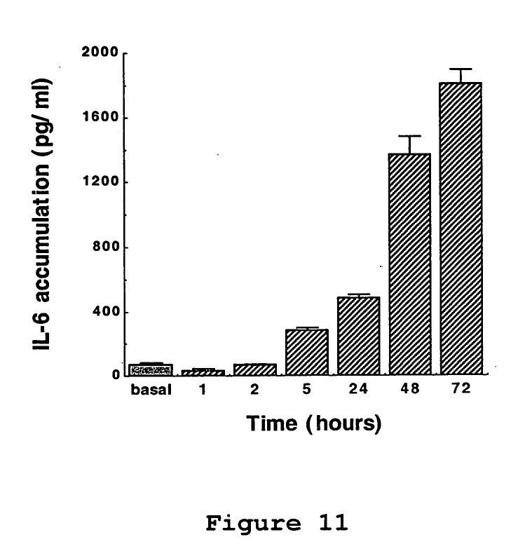

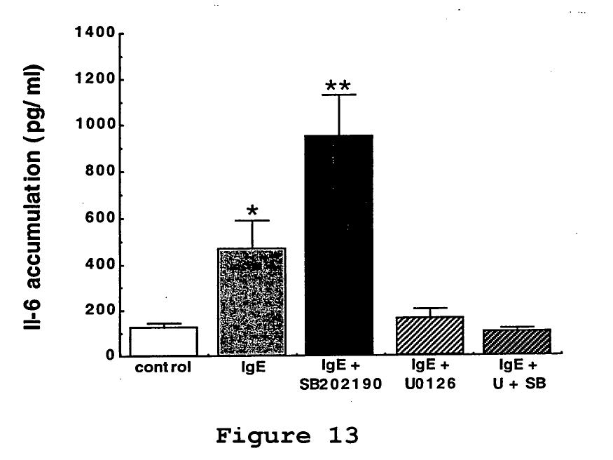

Previous studies have demonstrated that isolated airway smooth muscle (ASM) sensitized with IgE exhibits pro-asthmatic like changes in its constrictor and relaxant responsiveness, and that this phenomenon is attributed to induced proinflammatory cytokine release by the IgE sensitized ASM. To elucidate the signaling mechanism underlying IgE induced proinflammatory cytokine production by ASM, we examined the role of MAP kinase signaling pathways in IgE sensitized cultured human ASM (HASM) cells. In separate experiments we observed the following: HASM cells exposed for 48 hr to IgE immune complexes exhibited significantly enhanced (approximately 4 fold) release of the proinflammatory cytokine, IL 6, into the cell culture medium. See FIG. 11. This IgE-induced release of IL-6 was further significantly augmented (approximately 9 fold) when HASM cells were pretreated with the p38 MAP kinase inhibitor, SB-202190. In contrast to the the latter observation, concomitant pretreatment of HASM cells with the MEK1/2 selective inhibitor, U-0126, completely abrogated the IgE induced release of IL 6. See FIG. 13. Taken together these observations provide new evidence indicating that IgE-induced release of IL 6 from HASM cells is regulated by activation of both the MEK extracellular signal regulated kinases 1 and 2 (ERK1/2 and p38 MAP kinase signaling pathways.

The induced release of IL 6 from IgE exposed HASM cells is attributed to activation of the MEK ERK1/2 pathway. Since the release of IL 6 is potentiated by inhibition of p38 MAP kinase, this finding is consistent with a reported negative regulatory effect of p38 MAP kinase activation on the ERK1/2 signaling pathway.

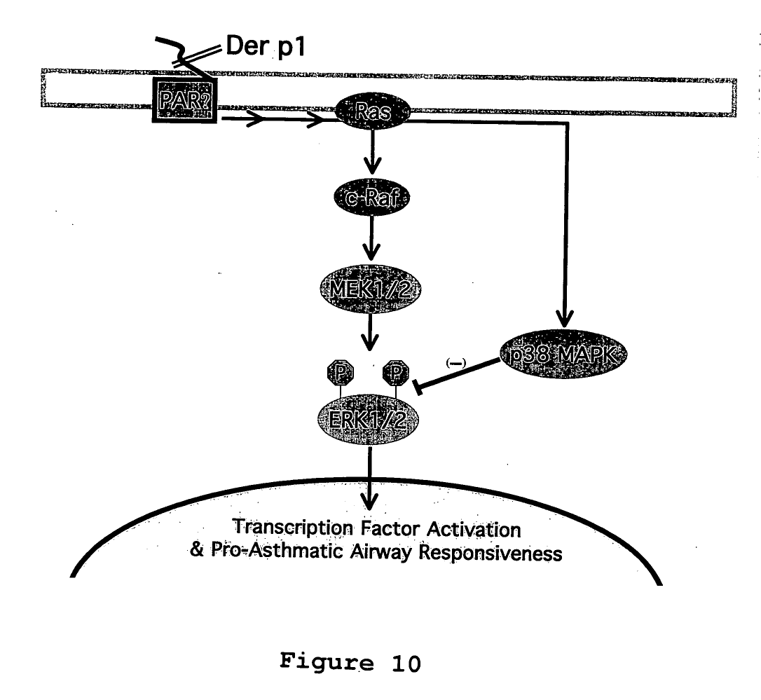

The findings presented in this example indicate that IgE induced proinflammatory cytokine release and changes in agonist responsiveness in ASM are regulated by dual activation of the MEK ERK1/2 and p38 MAP kinase signaling pathways. MEK ERK1/2 activation elicits the evoked changes in cytokine release and ASM responsiveness while these effects are attenuated by co-activated p38 MAP kinase which is secondary to its inhibition of ERK1/2 phosphorylation. See FIG. 10.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

1. A method for the treatment of asthma, comprising administration of an effective amount of a p38 agonist in a patient in need thereof.

2. The method of claim 1, wherein said agonist is anisomycin or derivative thereof.

3. The method of claim 1, wherein said agonist is administered systemically.

4. The method of claim 1, wherein said agonist is administered directly to the airway of said patient via inhalation.

5. The method of claim 1, wherein said patient has been exposed to housedust mite allergen.

6. The method of claim 5, wherein said allergen is the cytseine protease allergen, Der p1.

7. The method of claim 1, further comprising administration of an agent selected from the group consisting of corticosteroids, sodium cromolyn, methylxanthines, leukotriene modifiers and beta-adrenergic agents.

8. The method of claim 2, wherein said anisomycin derivative is selected from the group consisting of 4-O-dodecanoyl-3-O-carbamoyldeacetylanisomycin; 3-O-methylcarbamoyl-deacetylanisomycin; 4-O-acetyl-3-O-carbamoyldeacetylanisomycin; 4-O-hexanoyl-3-O-carbamoyldeacetylanisomycin; 4-O-heptanoyl-3-O-carbamoyldeacetylanisomycin; 3-O-methoxymethyldeacetylanisomycin and 3-O-carbamoyldeacytylanisomycin.

9. A method for identifying test compounds which act as agonists for p38 mitogen activated protein kinase (MAPK), comprising;
a) providing cells which express p38 and exhibit an inflammatory response in response to challenge with a pro-asthmatic agent;
b) challenging said cells with said stimulus in the presence and absence of said test compound;
c) determining the phosphorylation state of p38MAPK in the presence and absence of said test compound, compounds increasing the phosphorylation state of p38MAPK being identified as agonsts of p38MAPK.

10. The method of claim 9, wherein said cells are smooth muscle cells and tissue and wherein said method further comprises measuring constrictor and relaxation responses in the presence and absence of said test compound.

11. The method of claim 9, wherein said agent is selected from the group consisting of Der p1, lipopolysaccharide, IgE, rhinovirus, respiratory syncytial virus and parainfluenza virus.

12. The method of claim 9, wherein the phosphorylation state of p38MAPK is determined using Western blotting.

13. The method of claim 9, wherein said test compound is anisomycin or a derivative thereof.

14. The method of claim 9, further comprising determination of the phosphorylation state of EFK1/2.

15. The method of claim 9, further comprising determination of cytokine releases from said cells.

16. The method of claim 15, wherein said cytokine is IL-6.

17. The method of claim 9, wherein said cells comprise a co-culture of dendritic cells and antigen presenting cells.

18. A method for identifying test compounds which act as agonists for p38 comprising:
a) providing asthmatic mice;
b) administering an effective amount of said test compound to said mice for a period sufficient for agonist activity against p38 to occur, if any; and
c) determining the phosphorylation state of p38 in airway cells in the presence and absence of said test compound, compounds which increase the phosphorylation state of p38 relative to untreated control mice being identified as agonists of p38.

19. The method of claim 18, further comprising assessing whether said test compound reduces inflammation in the airways of said mice, said reduction in inflammation being correlated with a reduction in eosinophilia, airway mucus production and expression of VCAM-1.

20. The method of claim 18, wherein the phosphorylation state of p38MAPK is determined using Western blotting of airway cells removed from said mice after administration of said test compound.

21. The method of claim 18, wherein said test compound is anisomycin or a derivative thereof.

22. The method of claim 18, further comprising determination of the phosphorylation state of EFK1/2.

23. The method of claim 19, further comprising determination of cytokine release from said airway cells.

24. The method of claim 23, wherein said cytokine is IL-6.
